(19) United States
(12) Patent Application Publication
(10) Pub. No.: US 2026/0072149 A1
(43) Pub. Date: Mar. 12, 2026

(54) SYSTEMS AND METHODS FOR ULTRASOUND IMAGING WITH DYNAMIC MICROBEAMFORMERS

(71) Applicant: Exo Imaging, Inc., Santa Clara, CA (US)

(72) Inventors: Chad STEWARD, Sunnyvale, CA (US); Kutay USTUNER, Mountain View, CA (US)

(21) Appl. No.: 19/388,953

(22) Filed: Nov. 13, 2025

Related U.S. Application Data
(63) Continuation of application No. 17/709,953, filed on Mar. 31, 2022, now abandoned.

(51) Int. Cl. G01S 7/52; A61B 8/00; G01S 7/524; G01S 15/89

(52) U.S. Cl. CPC G01S 7/52044; G01S 7/5209; G01S 7/52095; G01S 7/524; G01S 15/892; G01S 15/8925; A61B 8/4494; G01S 15/8993

(57) ABSTRACT

An ultrasound imaging system can include an array of transducer elements, a plurality of receive circuits configured to provide one or more output signals, a plurality of delay circuits configured to output one or more delayed signals, and at least one multi-channel beamformer. The beamformer can receive representations of a plurality of microbeamformed signals and output at least one representation of a beamformed signal. The plurality of microbeamformed signals may represent a combination of delayed signals from the plurality of delay circuits. The plurality of delay circuits may be characterized by a plurality of time delay values. The plurality of time delay values may be controllable or adjustable such that one or more points-of-focus characterizing the microbeamformed signals can move along a line of sight.

### CROSS-REFERENCE TO RELATED APPLICATIONS

This application is a continuation of U.S. patent application Ser. No. 17/709,953, filed Mar. 31, 2022, which is herein incorporated by reference in its entirety.

### BACKGROUND

Non-intrusive imaging systems may be used to image internal tissue, bones, blood flow, or organs of human or animal body, or other objects of interest, such as a toy or a shipment package. Such systems and/or probes may require transmission of a signal into the body and the receiving of an emitted or reflected signal from the object or body part being imaged. In some cases, transducers or transceivers may be used to perform imaging, including imaging based on photo-acoustic and/or ultrasonic effects.

### SUMMARY

The present disclosure provides systems, devices, and methods for ultrasound imaging, including three-dimensional (3D) imaging based on ultrasound waves or other audio waves.

The present disclosure provides an ultrasound imaging system with a multi-stage receive beamformer. The first stage of the multi-stage receive beamformer may be a dynamically focused microbeamformer. The microbeamformer may comprise an array of transducer elements coupled to the inputs of multiple receive circuits. In some embodiments, the multiple receive circuit outputs may be coupled to the inputs of multiple delay circuits, each characterized by a time delay value and each providing a delayed signal as an output. In some cases, the time delay values characterizing the microbeamforming delay circuits can be controlled such that the points-of-focus characterizing the microbeamformed signals moves along a line of sight at a propagation rate. In some embodiments, the multiple delayed signals can be weighted and connected to the inputs of multiple summing amplifiers. In some embodiments, each summing amplifier can provide a microbeamformed signal representing a combination of delayed signals as an output. In some embodiments, multiple representations of the multiple microbeamformed signals can be coupled to the inputs of a second-stage multi-channel beamformer which provides a beamformed signal as an output.

In some preferred embodiments, the microbeamforming ultrasound imaging systems disclosed herein may be configured to control the time delay values used to form the microbeamformed signals such that the points-of-focus characterizing the microbeamformed signals substantially move along a line-of-sight at an expected propagation rate. This control of the time delay values can help to optimize image quality. In some embodiments, the time delay values used in a multi-channel beamformer can be controlled such that the point-of-focus of a fully beamformed output signal substantially moves along a line of sight. This control of the time delay values in the microbeamformer can provide a significant benefit over conventional systems that use time delay values that are chosen or set prior to transmission of an ultrasound pulse (e.g., to represent an expected difference in arrival time between elements within the group for echoes received from a single point-of-focus at a given steering angle and distance) and kept fixed while the received signal is being observed. Unlike conventional microbeamformers which can only focus at one point in time (corresponding to a single point-of-focus), the presently disclosed systems may permit a points-of-focus to move along a line-of-sight.

In one aspect, the present disclosure provides an ultrasound imaging system comprising an array of transducer elements; a plurality of receive circuits configured to provide one or more output signals; a plurality of delay circuits configured to output one or more delayed signals; and at least one multi-channel beamformer configured to (i) receive representations of a plurality of microbeamformed signals and (ii) output at least one representation of a beamformed signal.

In some embodiments, the plurality of receive circuits comprise at least one input coupled to at least one transducer element of the array of transducer element. In some embodiments, the plurality of delay circuits comprise one or more inputs coupled to at least one receive circuit of the plurality of receive circuits. In some embodiments, the plurality of delay circuits are characterized by a plurality of time delay values. In some embodiments, the plurality of time delay values are controllable or adjustable such that one or more points-of-focus characterizing the microbeamformed signals move along a line of sight. In some embodiments, the plurality of microbeamformed signals represent a combination of delayed signals from the plurality of delay circuits.

In some embodiments, at least one of the plurality of receive circuits comprises a time-gain-compensation circuit. In some embodiments, at least one of the plurality of delay circuits comprises an analog sample-and-hold delay circuit. In some embodiments, at least one of the plurality of delay circuits comprises an analog delay circuit. In some embodiments, the multi-channel beamformer comprises an analog multi-channel beamformer. In some embodiments, at least one of the plurality of delay circuits comprises an analog delay circuit. In some embodiments, the multi-channel beamformer comprises a digital multi-channel beamformer. In some embodiments, at least one of the plurality of delay circuits comprises a digital delay circuit. In some embodiments, the multi-channel beamformer comprises a digital multi-channel beamformer.

In some embodiments, the array of transducer elements comprises a one dimensional array. In some embodiments, the array of transducer elements comprises a two dimensional array. In some embodiments, the transducer elements comprise one or more PMUT elements. In some embodiments, the transducer elements comprise one or more CMUT elements.

In some embodiments, the points-of-focus characterizing the microbeamformed signals substantially move along the line of sight at a rate that is approximately half of a propagation rate of an ultrasound signal in a material being imaged. In some embodiments, the line of sight corresponds to an imaging path or at least a portion of an imaging region of interest.

In another aspect, the present disclosure provides an ultrasound imaging system comprising an array of transducer elements; a plurality of receive circuits configured to provide one or more output signals; a plurality of delay circuits configured to output one or more delayed signals; a plurality of delay calculator circuits configured to control or adjust one or more time delay values characterizing the plurality of delay circuits; and at least one multi-channel beamformer configured to (i) receive representations of a plurality of microbeamformed signals and (ii) output at least one representation of a beamformed signal.

In some embodiments, each of the plurality of receive circuits comprises at least one input coupled to at least one transducer element of the array of transducer element. In some embodiments, the plurality of delay circuits comprise one or more inputs coupled to at least one receive circuit of the plurality of receive circuits. In some embodiments, the plurality of microbeamformed signals represent a combination of delayed signals from the plurality of delay circuits.

In some embodiments, the plurality of delay calculator circuits are configured to control or adjust the one or more time delay values such that one or more points-of-focus characterizing the microbeamformed signals move along a line of sight at a propagation rate. In some embodiments, at least one delay calculator circuit of the plurality of delay calculator circuits is configured to calculate at least one time delay value by summing at least one constant term and at least one exponentially decaying term. In some embodiments, at least one delay calculator circuit of the plurality of delay calculator circuits is configured to use a linear interpolation algorithm to calculate the one or more time delay values. In some embodiments, at least one delay calculator circuit of the plurality of delay calculator circuits is configured to use a coordinate rotation digital computation (CORDIC) algorithm to calculate the one or more time delay values.

In some embodiments, at least one receive circuit of the plurality of receive circuits comprises a time-gain-compensation circuit. In some embodiments, at least one delay circuit of the plurality of delay circuits comprises an analog sample-and-hold delay circuit. In some embodiments, at least one delay circuit of the plurality of delay circuits comprises an analog delay circuit. In some embodiments, the multi-channel beamformer comprises an analog multi-channel beamformer. In some embodiments, at least one delay circuit of the plurality of delay circuits comprises an analog delay circuit. In some embodiments, the multi-channel beamformer comprises a digital multi-channel beamformer. In some embodiments, at least one delay circuit of the plurality of delay circuits comprises a digital delay circuit. In some embodiments, the multi-channel beamformer comprises a digital multi-channel beamformer.

In some embodiments, the array of transducer elements comprises a one dimensional array. In some embodiments, the array of transducer elements comprises a two dimensional array. In some embodiments, the array of transducer elements comprises a two dimensional array on a curved surface. In some embodiments, the transducer elements comprise one or more PMUT elements. In some embodiments, the transducer elements comprise one or more CMUT elements. In some embodiments, the transducer elements, the receive circuits, the delay circuits, and the delay calculator circuits are configured or physically arranged to have at least one common pitch in at least one dimension.

In some embodiments, the system may further comprise one or more converters. In some embodiments, the one or more converters comprise an analog-to-digital converter.

In another aspect, the present disclosure provides a method, comprising: (a) receiving one or more signals for ultrasound imaging of a target region; (b) generating one or more delayed signals based on (i) the one or more signals for ultrasound imaging and (ii) one or more time delay values; (c) aggregating the one or more delayed signals to form a plurality of microbeamformed signals; and (d) using a multi-channel beamformer to combine the plurality of microbeamformed signals to produce a beamformed signal. In some embodiments, the one or more time delay values are adjustable to move one or more points-of-focus characterizing the microbeamformed signals along a line of sight spanning at least a portion of the target region.

In some embodiments, the one or more delayed signals are aggregated by one or more summing amplifiers to form the plurality of microbeamformed signals.

In some embodiments, the method may further comprise, subsequent to (c), providing the plurality of microbeamformed signals to one or more converters. In some embodiments, the one or more converters comprise an analog-to-digital converter. In some embodiments, the one or more converters are configured to provide digital representations of the microbeamformed signals. In some embodiments, the multi-channel beamformer is configured to aggregate the digital representations of the microbeamformed signals to provide a digital representation of the beamformed signal.

In some embodiments, the multi-channel beamformer is configured to delay and combine the plurality of microbeamformed signals to produce the beamformed signal. In some embodiments, the one or more time delay values are adjusted using a plurality of delay calculator circuits. In some embodiments, the delayed signals are generated using one or more delay circuits. In some embodiments, the one or more delay circuits comprise an analog delay circuit. In some embodiments, the one or more delay circuits comprise a digital delay circuit.

In some embodiments, the one or more signals for ultrasound imaging are received from (i) an array of transducer elements or (ii) one or more receive circuits operatively coupled to the array of transducer elements. In some embodiments, the array of transducer elements comprises one or more PMUT elements. In some embodiments, the array of transducer elements comprises one or more CMUT elements. In some embodiments, the array of transducer elements comprises a one dimensional array. In some embodiments, the array of transducer elements comprises a two dimensional array.

In some embodiments, the points-of-focus characterizing the microbeamformed signals move along the line of sight at a rate that is approximately half of a propagation rate of an ultrasound signal in a material being imaged. In some embodiments, the line of sight corresponds to an imaging path or at least a portion of an imaging region of interest.

Another aspect of the present disclosure provides a non-transitory computer readable medium comprising machine executable code that, upon execution by one or more computer processors, implements any of the methods above or elsewhere herein.

Another aspect of the present disclosure provides a system comprising one or more computer processors and computer memory coupled thereto. The computer memory comprises machine executable code that, upon execution by the one or more computer processors, implements any of the methods above or elsewhere herein.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

A better understanding of the features and advantages of the present disclosure will be obtained by reference to the following detailed description that sets forth illustrative embodiments and the accompanying drawings.

FIG. 1 shows a block diagram of a microbeamforming ultrasound imaging system in accordance with some embodiments.

FIG. 2 shows a schematic of expected time-of-flight between one or more targets and one or more ultrasound transducer elements.

FIG. 3 shows an exemplary implementation of an analog sample-and-hold delay circuit.

FIG. 4 shows an implementation of a delay calculator circuit, in accordance with some embodiments.

FIG. 5 shows an exemplary schematic diagram of an ultrasonic imaging system, in accordance with some embodiments.

FIG. 6 shows an exemplary block diagram of an ultrasonic imaging system, in accordance with some embodiments.

FIG. 7 shows another exemplary schematic diagram of an ultrasonic imaging system, in accordance with some embodiments.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

### Certain Definitions

Unless otherwise defined, all technical terms used herein have the same meaning as commonly understood by one of ordinary skill in the art to which the present subject matter belongs.

As used herein, the singular forms “a,” “an,” and “the” include plural references unless the context clearly dictates otherwise. Any reference to “or” herein is intended to encompass “and/or” unless otherwise stated.

As used herein, the term “about” refers to an amount that is near the stated amount by about 10%, 5%, or 1%, including increments therein.

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

The term “real time” or “real-time,” as used interchangeably herein, generally refers to an event (e.g., an operation, a process, a method, a technique, a computation, a calculation, an analysis, a visualization, an optimization, etc.) that is performed using recently obtained (e.g., collected or received) data. In some cases, a real time event may be performed almost immediately or within a short enough time span, such as within at least 0.0001 millisecond (ms), 0.0005 ms, 0.001 ms, 0.005 ms, 0.01 ms, 0.05 ms, 0.1 ms, 0.5 ms, 1 ms, 5 ms, 0.01 seconds, 0.05 seconds, 0.1 seconds, 0.5 seconds, 1 second, or more. In some cases, a real time event may be performed almost immediately or within a short enough time span, such as within at most 1 second, 0.5 seconds, 0.1 seconds, 0.05 seconds, 0.01 seconds, 5 ms, 1 ms, 0.5 ms, 0.1 ms, 0.05 ms, 0.01 ms, 0.005 ms, 0.001 ms, 0.0005 ms, 0.0001 ms, or less.

### Ultrasound Imaging

The present disclosure provides systems and methods for ultrasound imaging. Ultrasound imaging may involve transmitting an ultrasound pulse and receiving a reflected signal at an array of transducer elements.

In one aspect, the present disclosure provides ultrasound imaging systems and, more specifically, electronically steered and focused ultrasound imaging systems. In another aspect, the present disclosure provides an ultrasound imaging system with improved control of steering and focus.

In some embodiments, the ultrasound imaging system may comprise an array of transducer elements. In some cases, the system can transmit an ultrasound pulse from the transducer array and receive the resulting reflected ultrasound energy by observing the outputs of the transducer elements over time. The elapsed time from transmit to receive can be representative of the distance from the transducer elements to a desired point-of-focus. In some embodiments, the ultrasound imaging system may comprise a receiver which can combine the outputs of the transducer elements into one or more beamformed or microbeamformed signals.

In one aspect, the present disclosure provides a system for ultrasound imaging. The system may comprise an array of transducer elements. In some cases, the system may comprise a plurality of receive circuits configured to provide one or more output signals. In some embodiments, the plurality of receive circuits may comprise at least one input coupled to at least one transducer element of the array of transducer element.

In some cases, the system may comprise a plurality of delay circuits configured to output one or more delayed signals. In some cases, the plurality of delay circuits may comprise one or more inputs coupled to at least one receive circuit of the plurality of receive circuits. In some cases, the plurality of delay circuits may be characterized by a plurality of time delay values.

In some cases, the system may comprise at least one multi-channel beamformer configured to (i) receive representations of a plurality of microbeamformed signals and (ii) output at least one representation of a beamformed signal. In some cases, the plurality of microbeamformed signals may represent a combination of delayed signals from the plurality of delay circuits. In some embodiments, the plurality of time delay values may be controllable or adjustable such that one or more points-of-focus characterizing the microbeamformed signals can move along a line of sight.

### Transducer Array

In some embodiments, the system may comprise a transducer array. The transducer array may comprise a plurality of elements for transmitting and/or receiving waves, signals, or pulses. The waves, signals, or pulses may comprise ultrasound waves, signals, or pulses.

In some embodiments, the ultrasound imaging system may comprise an array of transducer elements. A transducer element may be a piezoelectric transducer (PZT), a piezoelectric micromachined ultrasonic transducer (PMUT), or a capacitive micromachined ultrasonic transducer (CMUT). In some cases, the transducer elements may comprise one or more PMUT elements and/or one or more CMUT elements.

The array of transducer elements may comprise a one dimensional (1D) array or a two dimensional (2D) array. In some embodiments, the 2D array may comprise a 2D array on a curved surface.

In some embodiments, an array of transducer elements may be physically arranged in rows and/or columns in a repeating arrangement such as a rectangular grid or a hexagonal grid. In some cases, the array of transducer elements may comprise at least one common pitch representing the physical space between elements in at least one dimension.

### Receive Circuits

In some embodiments, the system may comprise one or more receive circuits. The plurality of receive circuits may comprise at least one input coupled to at least one transducer element of the array of transducer element. The plurality of receive circuits may be configured to generate or produce one or more output signals. The one or more output signals may correspond to one or more signals, waves, or pulses transmitted or received by the transducer array or any one or more elements thereof.

In some embodiments, the ultrasound imaging system may comprise receive circuits having at least one input coupled to a transducer element and providing at least one output. In some cases, the receive circuits may provide one or more analog signals or one or more digital signals as an output.

In some cases, the receive circuits may comprise switches, amplifiers, sample-and-hold circuits, attenuators, time-gain compensation (TGC) circuits, ADCs, and/or digital logic. The amplifiers may comprise, for example, low-noise amplifiers (LNAs), variable-gain amplifiers (VGAs), fixed-gain amplifiers, summing amplifiers, single-ended amplifiers, or differential amplifiers. In some cases, at least one of the plurality of receive circuits may comprise a time-gain-compensation circuit. The time-gain compensation (TGC) circuit may be configured to receive a signal as an input and may provide an output which substantially represents an input multiplied by a TGC gain. In some embodiments, the TGC gain may be controlled to change with time. In some cases, the TGC gain may be greater than or equal to one (amplification) or less than or equal to one (attenuation). In some cases, the TGC circuit may comprise variable gain amplifiers (VGAs), fixed gain amplifiers, attenuators, resistors, or capacitors. In some embodiments, the ultrasound imaging system may comprise TGC circuits having TGC gains that can be controlled to compensate for an expected change in amplitude of a received ultrasound signal with time.

### Delay Circuits

In some embodiments, the system may comprise a plurality of delay circuits. The plurality of delay circuits may comprise one or more inputs coupled to at least one receive circuit of the plurality of receive circuits. The plurality of delay circuits may be configured to output one or more delayed signals. The delayed signal may correspond to one or more output signals received from the plurality of receive circuits. The delay signal may comprise at least one signal to which a time delay has been applied. The at least one signal may comprise the one or more output signals received from the plurality of receive circuits. In some cases, the plurality of delay circuits may be characterized by a plurality of time delay values. The time delay values may be dynamically adjustable.

In some cases, at least one of the plurality of delay circuits comprises an analog sample-and-hold delay circuit. In some cases, at least one of the plurality of delay circuits may comprise an analog delay circuit (e.g., in instances where a multi-channel beamformer comprising an analog multi-channel beamformer is used). In some cases, at least one of the plurality of delay circuits may comprise an analog delay circuit (e.g., in instances where a multi-channel beamformer comprising a digital multi-channel beamformer is used). In some cases, at least one of the plurality of delay circuits may comprise a digital delay circuit (e.g., in instances where a multi-channel beamformer comprising a digital multi-channel beamformer is used).

In some embodiments, the system may comprise one or more delay circuits. The one or more delay circuits may have at least one input and may be characterized by a time delay value (TDELAY). In some cases, the one or more delay circuits may provide at least one delayed signal as an output, such that at a time (t) the delayed signal substantially represents the input at an earlier time (t-TDELAY).

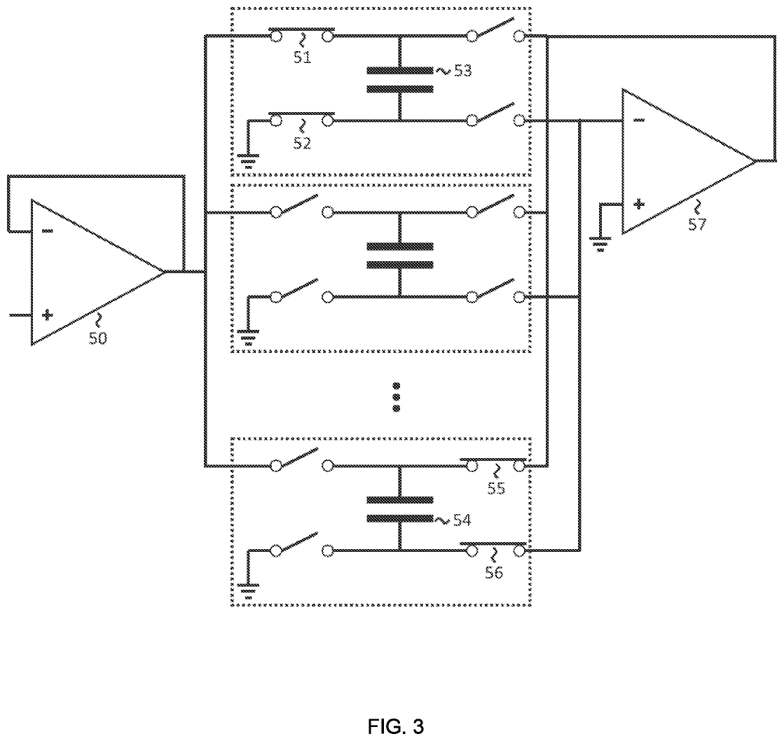

In some embodiments, the delay circuit may be an analog delay circuit which receives an analog signal as an input and provides an analog signal as an output. The analog delay circuit may be implemented using a sample-and-hold circuit such as the circuit illustrated in FIG. 3. In some cases, the sample-and-hold delay circuit may store a value representative of an input signal on one or more capacitors and may use the capacitors to provide a delayed signal as an output.

In some embodiments, analog signals provided as outputs from analog delay circuits may be combined by connecting capacitors together, which can have the effect of summing or averaging values stored on the capacitors. In some cases, analog signals provided as outputs from delay circuits may be combined by coupling the analog signals to the inputs of a summing amplifier circuit. The summing amplifier circuit may comprise resistors or capacitors coupled to amplifier inputs or outputs.

In some embodiments, the delay circuit may comprise a digital delay circuit which receives a digital signal as an input and provides a digital signal as an output. The digital delay circuit may comprise a first-in first-out buffer (FIFO) having an input address specified by a write pointer value and an output address specified by a read pointer value. Read and write pointer values may both be controlled to increment at a predetermined rate. In some cases, the delay value of a digital delay circuit may be adjusted by controlling the difference between a read pointer value and a write pointer value.

In some embodiments, the digital delay circuit may comprise a multi-bit parallel shift register and a multiplexer. The input of the shift register may be coupled to a digital input signal of the digital delay circuit. In some cases, data inputs of the multiplexer may be coupled to the contents of the shift register. The outputs of the multiplexer may provide a delayed digital signal at the output of the digital delay circuit. In some embodiments, the delay value of a digital delay circuit may be controlled by select inputs of the multiplexer.

In some cases, the digital delay circuit may comprise flip-flops, latches, or random-access memory (RAM) circuits. In some cases, digital signals provided as outputs from the digital delay circuits may be combined by adding representations of the digital signals together, which can have the effect of summing or averaging values represented by the digital signals.

### Time Delay Values

In some preferred embodiments, the microbeamforming ultrasound imaging systems disclosed herein may be configured to control the time delay values used to form the microbeamformed signals such that the points-of-focus characterizing the microbeamformed signals substantially move along a line-of-sight at an expected propagation rate. This control of the time delay values can help to optimize image quality. In some embodiments, the time delay values used in a multi-channel beamformer can be controlled such that the point-of-focus of a fully beamformed output signal substantially moves along a line of sight. This control of the time delay values can provide a significant benefit over conventional systems that use time delay values that are chosen or set prior to transmission of an ultrasound pulse (e.g., to represent an expected difference in arrival time between elements within the group for echoes received from a single point-of-focus at a given steering angle and distance) and kept fixed while the received signal is being observed. Unlike many conventional systems which can only focus at one point in time (corresponding to a single point-of-focus), the presently disclosed systems permit a points-of-focus to move along a line-of-sight.

In some cases, it may be desirable to create one or more steered and focused beamformed signals by delaying signals received at transducer elements within the transducer array and combining the delayed signals together. The value of the time delays may be chosen to compensate for the difference in expected arrival time for various elements in the transducer array. In some cases, the delayed signals may be weighted equally or differently when combined. In some cases, the delayed signals may be combined by summing (delay-and-sum) or averaging them together to create one or more beamformed signals.

### Point of Focus/Line of Sight

As described above, in some embodiments, the plurality of time delay values may be controllable or adjustable such that one or more points-of-focus characterizing the microbeamformed signals move along a line of sight. In some embodiments, the points-of-focus characterizing the microbeamformed signals may move along the line of sight at a rate that is approximately half of a propagation rate of an ultrasound signal in a material being imaged.

In some embodiments, the line of sight may correspond to an imaging path or at least a portion of an imaging region of interest. The imaging path may correspond to a scanning pattern or a scanning direction. In some cases, the scanning pattern may be defined by or associated with a specific imaging location or region or field of view. In some cases, the scanning direction may correspond to an imaging vector (i.e., a vector or a series of vectors defining a path along which imaging can be performed). In some cases, the line of sight may be associated with an imaging line. In some cases, the imaging line may correspond to a column or row or other linear or non-linear series of pixels in an image. In some cases, the imaging line (also referred to herein as a scan line) may correspond to at least a portion of the imaging vector(s) along which imaging is performed.

In some embodiments, the line of sight may correspond to a scan line of an image. As used herein, a scan line may correspond to or represent a portion of a frame representing an image. To form a frame, a transducer can focus and/or transmit waves (e.g., acoustic or pressure waves) from different piezoelectric elements to a particular focal point. The reflected signals collected by these piezoelectric elements can be received, delayed, weighted, and summed to form a scan line. The focal point of interest can be changed to different parts of the frame, and the process can be repeated until an entire frame comprising a plurality of scan lines is generated.

In some embodiments, the line of sight may correspond to an imaging path of the ultrasound imaging system. The imaging path may cover a target location or region or field of view of interest. The imaging path may comprise one or more imaging vectors (i.e., a vector or a series of vectors defining a path along which imaging can be performed). The imaging path may comprise one or more linear portions and/or one or more curved portions.

In some embodiments, a line of sight may be chosen to originate at the center of the transducer array. In other embodiments, the line of sight may be chosen to originate at another point inside or outside the transducer array, behind the transducer, array or in front of the transducer array. In some cases, the line of sight may be angled directly forward (e.g., perpendicular to the transducer array). In other cases, the line of sight may be angled away from the perpendicular in one or more dimensions.

### Propagation Rate

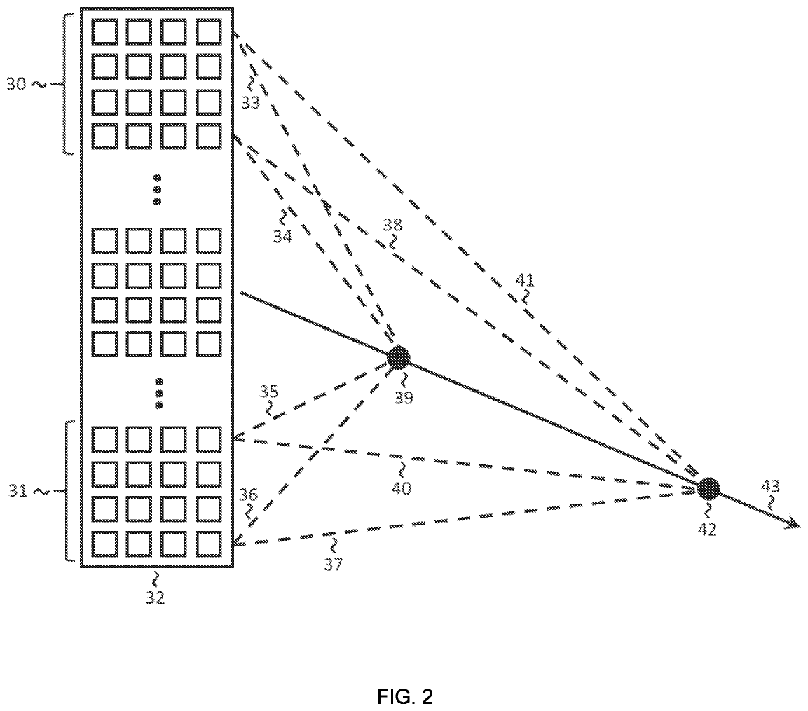

Referring to FIG. 2, at a given point in time, the value of a beamformed signal may represent the ultrasound signal reflected from a target at a point-of-focus, such as point-of focus 39 or 42. This point-of-focus 39 or 42 may change over time, and in some cases can substantially move along a line of sight 43 at a propagation rate. This propagation rate may be approximately half of the propagation rate of the ultrasound signal in the material being imaged. For example, in human tissue a point of focus may move along a line of sight at approximately 770 meters per second.

In some cases, an ultrasound signal reflected from a target located at a desired point-of-focus may arrive at each transducer element at a different time, as shown in FIG. 2. This difference in arrival times may be determined by the propagation rate of the ultrasound signal, the locations of elements within the transducer array, and/or the angle and distance to the desired point-of-focus.

### Delay Calculator

In some embodiments, the ultrasound imaging system may comprise one or more delay calculators. The delay calculator may be implemented in hardware (e.g., as a delay calculator circuit) or in software. In some embodiments, the delay calculator circuit may comprise adders or multipliers. In some embodiments, the delay calculator circuit may use a coordinate rotation digital computation (CORDIC) algorithm. As used herein, a CORDIC algorithm may comprise an algorithm that calculates trigonometric functions, hyperbolic functions, square roots, multiplications, divisions, and exponentials and logarithms with arbitrary base, typically converging with one digit (or bit) per iteration. The CORDIC algorithm may be used in instances where no hardware multiplier is available (e.g. in simple microcontrollers and FPGAs), as the only operations it requires are additions, subtractions, bitshift and lookup tables.

In some cases, the delay calculator may be used to compute one or more expected times-of-flight from a point-of-focus to a location. In some cases, the delay calculator may be used to compute one or more expected differences between times-of-flight from a point-of-focus to multiple locations. In some cases, the delay calculator may be used to control one or more time delay values characterizing one or more delay circuits as described elsewhere herein. In some cases, the delay calculator may be used to control time delay values so that points-of-focus of beamformed signals and/or microbeamformed signals substantially move along a line-of-sight at a propagation rate.

In some embodiments, the delay calculator circuit may calculate a time-of-flight between two points based upon a distance between the points in 2 dimensions (X, Y) or 3 dimensions (X, Y, Z). The time-of-flight may be proportional to the square root of the quantity X2+Y2+Z2.

In some embodiments, the delay calculator circuit may use interpolation to approximate times-of-flight or to approximate differences between times-of-flight. In some cases, the delay calculator may use interpolation across time, physical space, beam origin, or beam angle. The delay calculator may use linear or non-linear interpolation. In some cases, the delay calculator circuit may calculate a sum of a constant term and an exponentially decaying term to perform a non-linear interpolation across time.

In some cases, at least some of the receive circuits, delay circuits, or delay calculator circuits described herein can be physically located away from the transducer array, or arranged in a different configuration than the transducer array. In some cases, at least some of these circuits may be located adjacent or proximal to the transducer array and physically arranged to have at least one pitch in common with the transducer array, which can help to reduce size, cost, and power requirements.

### Converters

In some embodiments, the ultrasound imaging system may comprise one or more analog-to-digital converter (ADC) circuits which can be configured to receive at least one analog signal as an input and provide at least one digital representation of the input as an output. In some cases, the one or more ADC circuits may be implemented as a successive-approximation ADC, a pipelined ADC, a sigma-delta ADC, a flash ADC, a Nyquist ADC, or an oversampling ADC. In some cases, the ultrasound imaging system may comprise ADCs which receive analog microbeamformed signals as inputs and provide digital representations of microbeamformed signals as outputs.

### Beamforming

The ultrasound imaging systems disclosed herein may be configured to combine information from multiple signals (e.g., raw signals and/or beamformed signals) to form images representing the ultrasound signal reflected from a range of locations. These images may be 1D, 2D, or 3D pictures or videos, or may take the form of charts or graphs. These images may be monochromatic (black-and-white) or color images. These images may represent human tissue or other materials. These images may include information about density, size, velocity, or fluid flow. These images may be representative of the magnitude, phase, harmonic content, or other properties of the received ultrasound signals.

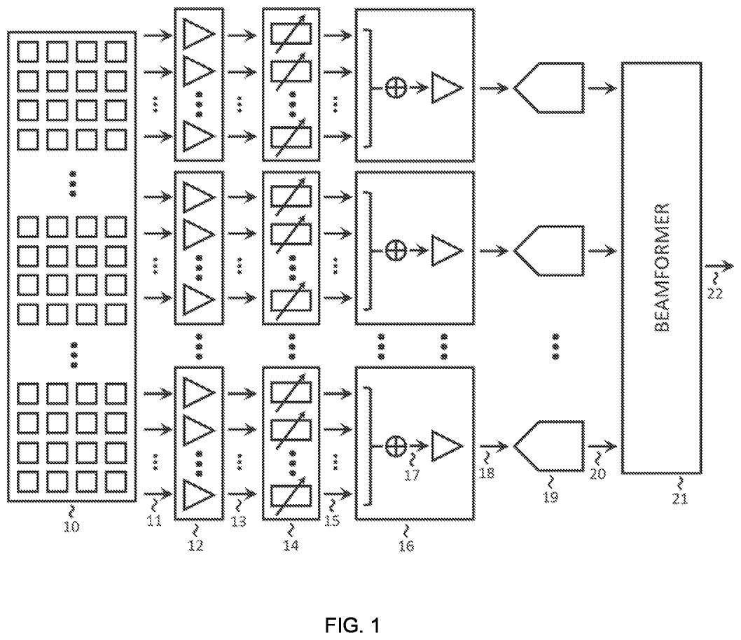

The ultrasound imaging systems described herein may perform or implement beamforming delay-and-sum operations in multiple steps (e.g., by using a micro-beamforming ultrasound system) to reduce (1) the size/footprint of the imaging system, (2) the cost of the imaging system, and/or (3) the power requirements of the imaging system. A block diagram of a micro-beamforming ultrasound imaging system in accordance with the present disclosure is shown in FIG. 1.

In some cases, the system may first delay and combine a number of signals from one or more groups of elements (e.g., localized elements) into a smaller number of microbeamformed signals. The microbeamformed signals may be provided as inputs to a multi-channel beamformer which may delay and combine the microbeamformed signals, for example using further delay-and-sum operations, to form one or more fully beamformed signals. Such a multi-channel beamformer may be configured to delay each microbeamformed signal to compensate for the difference between expected arrival time for each group of elements represented by each microbeamformed signal. This delay may produce a beamformed signal that can be steered and/or focused to a given point-of-focus at a given time.

In some embodiments, the ultrasound imaging system may comprise a multi-channel beamformer which may receive representations of multiple microbeamformed signals as inputs and may provide at least one representation of a beamformed signal as an output. The multiple microbeamformed signals may represent a combination of signals from multiple delay circuits.

In some cases, the multi-channel beamformer may comprise an analog multi-channel beamformer receiving analog signals as inputs and providing at least one analog signal as an output. In some cases, the multi-channel beamformer may comprise a digital multi-channel beamformer receiving digital signals as inputs and providing at least one digital signal as an output. The digital multi-channel beamformer may comprise memory elements and digital circuits implemented in an integrated circuit or a programmable logic device such as a field programmable gate array (FPGA). The digital multi-channel beamformer may comprise a microprocessor or microcontroller. The digital multi-channel beamformer may be implemented in hardware or software.

### Additional Embodiments

In another aspect, the present disclosure provides an ultrasound imaging system. The ultrasound imaging system may comprise an array of transducer elements. In some cases, the array of transducer elements may comprise a one dimensional array. In some cases, the array of transducer elements may comprise a two dimensional array, such as, for example, a two dimensional array on a curved surface. In some cases, the transducer elements may comprise one or more PMUT elements. In some cases, the transducer elements may comprise one or more CMUT elements.

In some embodiments, the imaging system may comprise a plurality of receive circuits configured to provide one or more output signals. In some cases, each of the plurality of receive circuits may comprise at least one input coupled to at least one transducer element of the array of transducer element. In some cases, at least one receive circuit of the plurality of receive circuits may comprise a time-gain-compensation circuit.

In some embodiments, the system may comprise a plurality of delay circuits configured to output one or more delayed signals. In some cases, the plurality of delay circuits may comprise one or more inputs coupled to at least one receive circuit of the plurality of receive circuits.

In some cases, at least one delay circuit of the plurality of delay circuits may comprise an analog sample-and-hold delay circuit. In some cases, at least one delay circuit of the plurality of delay circuits may comprise an analog delay circuit, for example, in some cases where a multi-channel beamformer comprising an analog multi-channel beamformer is used or in other cases where a multi-channel beamformer comprising a digital multi-channel beamformer is used. In some cases, at least one delay circuit of the plurality of delay circuits may comprise a digital delay circuit (e.g., in cases where a multi-channel beamformer comprising a digital multi-channel beamformer is used).

In some embodiments, the system may comprise a plurality of delay calculator circuits configured to control or adjust one or more time delay values characterizing the plurality of delay circuits. In some cases, the plurality of delay calculator circuits may be configured to control or adjust the one or more time delay values such that one or more points-of-focus characterizing the microbeamformed signals move along a line of sight at a propagation rate. In some cases, at least one delay calculator circuit of the plurality of delay calculator circuits may be configured to calculate at least one time delay value by summing at least one constant term and at least one exponentially decaying term. In some cases, at least one delay calculator circuit of the plurality of delay calculator circuits may be configured to use a linear interpolation algorithm to calculate the one or more time delay values. In some cases, at least one delay calculator circuit of the plurality of delay calculator circuits may be configured to use a coordinate rotation digital computation (CORDIC) algorithm to calculate the one or more time delay values.

In some embodiments, the system may comprise at least one multi-channel beamformer configured to (i) receive representations of a plurality of microbeamformed signals and (ii) output at least one representation of a beamformed signal. In some cases, the plurality of microbeamformed signals may represent a combination of delayed signals from the plurality of delay circuits.

In some cases, the imaging system may further comprise one or more converters. The one or more converters may comprise, for example, an analog-to-digital converter.

In any of the embodiments described herein, the transducer elements, the receive circuits, the delay circuits, and the delay calculator circuits may be configured or physically arranged to have at least one common pitch in at least one dimension.

### Imaging Method

In another aspect, the present disclosure provides a method for imaging. The method may comprise (a) receiving one or more signals for ultrasound imaging of a target region.

In some embodiments, the method may comprise (b) generating one or more delayed signals based on (i) the one or more signals for ultrasound imaging and (ii) one or more time delay values. In some cases, the one or more time delay values may be adjustable to move one or more points-of-focus characterizing the microbeamformed signals along a line of sight spanning at least a portion of the target region.

In some embodiments, the method may comprise (c) aggregating the one or more delayed signals to form a plurality of microbeamformed signals. In some embodiments, the method may comprise (d) using a multi-channel beamformer to combine the plurality of microbeamformed signals to produce a beamformed signal.

In some embodiments, the one or more delayed signals may be aggregated by one or more summing amplifiers to form the plurality of microbeamformed signals.

In some embodiments, the method may further comprise, subsequent to (c), providing the plurality of microbeamformed signals to one or more converters. In some embodiments, the one or more converters may comprise an analog-to-digital converter. In some embodiments, the one or more converters may be configured to provide digital representations of the microbeamformed signals.

In some embodiments, the multi-channel beamformer may be configured to aggregate the digital representations of the microbeamformed signals to provide a digital representation of the beamformed signal. In some embodiments, the multi-channel beamformer may be configured to delay and combine the plurality of microbeamformed signals to produce the beamformed signal.

In some embodiments, the one or more time delay values may be adjusted using a plurality of delay calculator circuits. In some embodiments, the plurality of delay circuits comprises an analog delay circuit and/or a digital delay circuit.

In some embodiments, the one or more signals for ultrasound imaging may be received from (i) an array of transducer elements or (ii) one or more receive circuits operatively coupled to the array of transducer elements. In some embodiments, the array of transducer elements may comprise one or more PMUT elements and/or one or more CMUT elements. In some embodiments, the array of transducer elements may comprise a one dimensional array, or a two dimensional array, such as a two dimensional array on a curved surface.

In some embodiments, the points-of-focus characterizing the microbeamformed signals may move along the line of sight at a rate that is approximately half of a propagation rate of an ultrasound signal in a material being imaged. In some embodiments, the line of sight may correspond to an imaging path or at least a portion of an imaging region of interest.

### Examples

In one aspect, the present disclosure provides a microbeamforming ultrasound imaging system. The microbeamforming ultrasound imaging system may comprise an array of transducer elements coupled to the inputs of multiple receive circuits. In some embodiments, the multiple receive circuit outputs may be coupled to the inputs of multiple delay circuits, each characterized by a time delay value and each providing a delayed signal as an output. In some cases, the time delay values characterizing the microbeamforming delay circuits can be controlled such that the points-of-focus characterizing the microbeamformed signals moves along a line of sight at a propagation rate. In some embodiments, the multiple delayed signals can be connected to the inputs of multiple summing amplifiers. In some embodiments, each summing amplifier can provide a microbeamformed signal representing a combination of delayed signals as an output. In some embodiments, multiple representations of the multiple microbeamformed signals can be coupled to the inputs of a multi-channel beamformer which provides a beamformed signal as an output.

In some embodiments, the transducer elements can be coupled to an array of receive circuits. The receive circuit outputs can be passed through an array of delay circuits characterized by time delay values. The delay circuit outputs can be combined to form multiple microbeamformed signals characterized by a point-of-focus determined by the time delay values. The microbeamformed signals can be coupled to a multichannel beamformer so that they can be further delayed and combined to form a beamformed signal. The time delay values can be controlled in real time after each transmitted ultrasound pulse to move the point-of-focus along a desired line of sight at the expected propagation rate of the received ultrasonic echo.

FIG. 1 illustrates an example of a microbeamforming ultrasound imaging system. The microbeamforming ultrasound imaging system may comprise an array of transducer elements 10 connected to multiple receive circuits 12. The multiple receive circuits 12 may receive one or more outputs 11 from the array of transducer elements 10. The one or more outputs 11 may correspond to one or more signals received by the array of transducer elements 10.

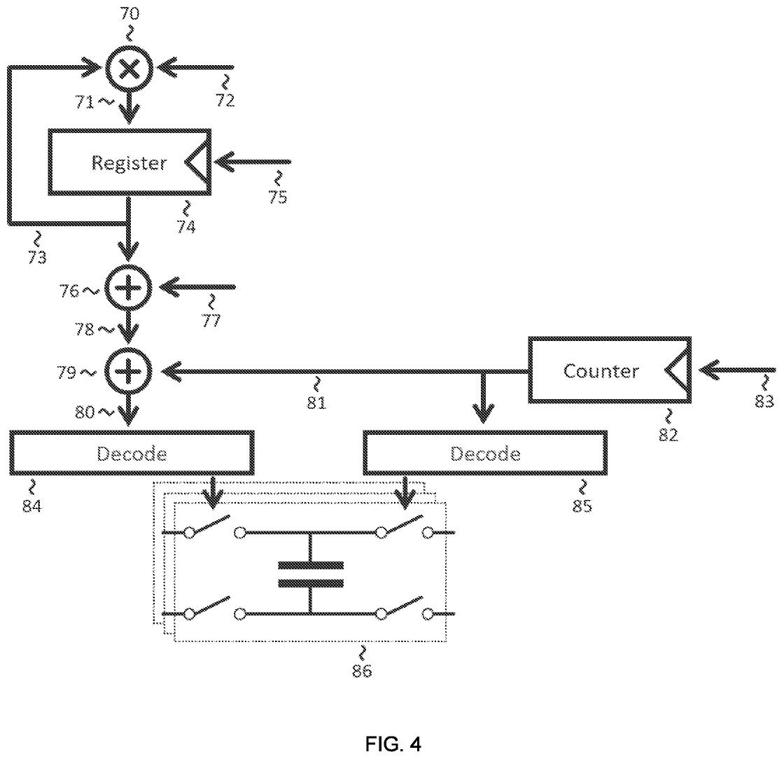

In some embodiments, the receive circuit outputs 13 may be connected to the inputs of multiple delay circuits 14, each characterized by a time delay value and each providing a delayed signal 15 at its output. The time delay values characterizing the delay circuits 14 may be substantially controlled by multiple delay calculator circuits as shown in FIG. 4.

In some embodiments, the delayed signals 15 may be connected to the inputs of multiple summing amplifiers 16. The summing amplifiers may be configured to combine groups of the delayed signals to form multiple microbeamformed signals 18.

In some embodiments, the microbeamformed signals 18 may be connected to the inputs of multiple analog-to-digital converters (ADCs) 19, each providing at least one of multiple digital representations 20 of the microbeamformed signals 18. In some cases, the digital representations 20 of the microbeamformed signals may be coupled to the inputs of a multi-channel beamformer circuit 21 which may delay and combine them to provide a digital representation of a beamformed signal 22.

As illustrated in FIG. 2, the time delay values characterizing the microbeamforming delay circuits 14 may be controlled such that points-of-focus 39, 42 characterizing the microbeamformed signals 18 substantially move along a line of sight 43 at a propagation rate.

In a preferred embodiment, the transducer elements 10, receive circuits 12, delay circuits 14, and delay calculator circuits may be physically arranged to have at least one common pitch in at least one dimension.

FIG. 4 shows a preferred embodiment of a delay calculator circuit for controlling a time-delay-value characterizing an analog delay circuit. In some cases, the delay calculator circuit may use non-linear interpolation. In some cases, the delay calculator circuit may calculate an exponentially decaying term 73 by repeatedly multiplying a stored value of the exponentially decaying term 73 by a fractional value 72. In some cases, the delay calculator circuit may calculate a sum 78 of the exponentially decaying term 73 and a constant term 77. The sum 78 may control a time-delay-value (TDELAY) characterizing an analog delay circuit 86. In one non-limiting embodiments, the analog delay circuit 86 may comprise an analog sample-and-hold delay circuit of the type illustrated in FIG. 3.

In some embodiments, a counter 82 may provide a value 81 which may control connections of capacitors 53 and 54 to an output amplifier 57. In some cases, an adder 79 may provide a value 80 which may control connections of the capacitors 53 and 54 of the delay circuit shown in FIG. 3 to an input amplifier 50 of the delay circuit. In some cases, the output amplifier 57 of the delay circuit may provide an output which at a time (t) is representative of the value of an input to the input amplifier 50 at a previous time (t-TDELAY). As described elsewhere herein, the time-delay-value (TDELAY) can be controlled, adjusted, or otherwise modified by the delay calculator circuit.

### Ultrasound Imaging System—Signal Types Used

In any of the embodiments described herein, the ultrasound imaging system may use various representations of ultrasound signals, input signals, output signals, delayed signals, microbeamformed signals, and/or fully beamformed signals.

In some cases, the ultrasound imaging system may use analog signals represented by one or more voltages, currents, impedances, pressures, or states of charge. The analog signals may comprise, for example, single-ended signals (such as a single voltage or current) or differential signals (such as the difference between a pair of voltages or currents).

In some cases, the ultrasound imaging system may use digital signals. The digital signals may be represented by multi-bit parallel binary signals or bit-serial binary signals.

In some cases, the ultrasound imaging system may use one or more analog signals and/or one or more digital signals. In some cases, the ultrasound imaging system may comprise one or more converters for converting between analog and digital signals, or between digital and analog signals.

### Ultrasound Imager/Imaging Probe

The systems and methods described herein may be used compatibly with any type of imaging device, including optical and acoustic imaging devices. In some cases, the optical imaging devices may comprise cameras, CMOS sensors, CCD sensors, or any other type of sensor configured for imaging based on optical signals. In some cases, the acoustic imaging devices may comprise ultrasound imaging devices. In some non-limiting embodiments, the imaging devices may be configured as handheld ultrasound imaging probes.

Ultrasound imaging (sonography) uses high-frequency sound waves to view inside the body. Because ultrasound images are captured in real-time, they can show movement of the body's internal organs as well as blood flowing through the blood vessels. The sound waves can also be used to create and display images of internal body structures such as tendons, muscles, joints, blood vessels, and internal organs.

To perform imaging, the imaging device transmits a signal into the body and receives a reflected signal from the body part being imaged. Types of imaging devices include transducers, which may also be referred to as transceivers or imagers, and which may be based on either photo-acoustic or ultrasonic effects. Such transducers can be used for imaging as well as other applications. For example, transducers can be used in medical imaging to view anatomy of tissue or other organs in a body. Transducers can also be used in industrial applications such as materials testing or therapeutic applications such as local tissue heating of HIFU based surgery. When imaging a target and measuring movement of the target, such as flow velocity and direction blood, Doppler measurements techniques are used. Doppler techniques are also applicable for industrial applications to measure flow rates, such as fluid or gas flow in pipes. The Doppler measurements may be based on the difference between transmitted and reflected wave frequencies due to relative motion between the source and the object. The frequency shift is proportional to the movement speed between the transducer and the object. This effect is exploited in ultrasound imaging to determine blood flow velocity and direction.

In some embodiments, the transducer elements described herein (e.g., pMUT elements, cMUT elements, etc.) may be interchangeably referred to as transceiver elements. In some cases, the transducer elements described herein may comprise piezoelectric elements or piezo elements. In some embodiments, the transducer elements described herein may include one or more of: a substrate, a membrane suspending from the substrate; a bottom electrode disposed on the membrane; a piezoelectric layer disposed on the bottom electrode; and one or more top electrodes disposed on the piezoelectric layer.

For ultrasound imaging, transducers can be used to transmit an ultrasonic beam towards the target to be imaged. A reflected waveform is received by the transducer, converted to an electrical signal and with further signal processing, an image is created. Velocity and direction of flow may be measured using an array of micro-machined ultrasonic transducers (MUTs).

The ultrasound devices disclosed herein may be configured for B-mode imaging. B-mode imaging for anatomy is a two-dimensional ultrasound image display composed of dots representing the ultrasound echoes. The brightness of each dot can be determined by the amplitude of the returned echo signal, allowing for visualization and quantification of anatomical structures, as well as for the visualization of diagnostic and therapeutic procedures. Usually, the B-mode image bears a close resemblance to the actual anatomy of a cutout view in the same plane. In B-mode imaging, a transducer is first placed in a transmit mode and then placed in receive mode to receive echoes from the target. The echoes are signal processed into anatomy images. The transducer elements are programmable such that they can be either in transmit mode or in receive mode, but not simultaneously.

The ultrasound devices disclosed herein may be configured for color Doppler imaging. The use of color flow Doppler, color Doppler imaging, or simply color Doppler allows the visualization of flow direction and velocity for blood in an artery or vein within a user defined area. A region of interest is defined, and the Doppler shifts of returning ultrasound waves are color-coded based on average velocity and direction. Sometimes these images are overlapped (co-imaged) with anatomy images in B-mode scan to present a more intuitive feel of flow relative to anatomy being viewed. Doppler imaging can also be PW Doppler so that the range and velocity of flow is determined, but maximum flow rate is dependent on pulse repetition frequency used, otherwise images are aliased making higher velocities look like lower velocities. Doppler shift can be measured from an ensemble of waves received to measure flow velocity using PW mode of Doppler imaging. CW Doppler is a continuous imaging technique where aliasing is avoided through continuous transmitting from one transducer element while receiving echoes from another transducer element. In a programmable instrument, both pulsed and continuous techniques can be implemented as discussed later. PW and Color Doppler may use a selected number of elements in an array. First, the elements are placed in a transmit mode and after echoes have returned, the elements are placed in a receive mode where the received signal is processed for Doppler signal imaging. For CW Doppler, at least two different elements are utilized, where each element is in transmit mode while the other element is in receive mode continuously.

The ultrasound imaging devices disclosed herein may be configured for two-dimensional (2D) imaging and/or three-dimensional (3D) imaging. In some embodiments, the ultrasound imaging devices may utilize one or more arrays of MEMS (micro-electromechanical system) ultrasound transducers such as pMUTs (piezoelectric micromachined ultrasound transducers) and/or cMUTs (capacitive micromachined ultrasound transducers). The micromachined ultrasonic transducers (MUTs) may be arranged in a lateral array. In some cases, the MUTs may be arranged in a regular or symmetric configuration. In other cases, the MUTs may be arranged in a staggered or asymmetric configuration. In some cases, the transducer elements may be arranged in a rectangular grid. In other cases, the elements can be arranged in a circular configuration, a rhombus (equilateral parallelogram), a hexagon, an annular shape, or an arbitrary grid, for example. The arrays can be on a curved surface as well as planar arrays.

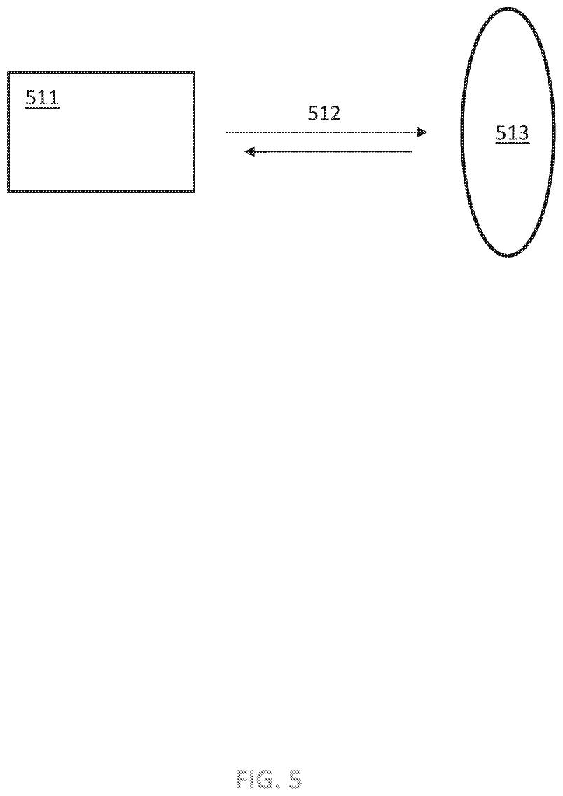

FIG. 5 illustrates an exemplary system 511 that may be used for imaging (e.g., ultrasound imaging) of a target region 513. The system 511 may comprise an imaging device (e.g., any of the imaging devices shown or described herein). The imaging device may be configured to transmit and receive one or more signals 512. The one or more signals 512 may comprise signals that are transmitted to and/or reflected from the target region 513.

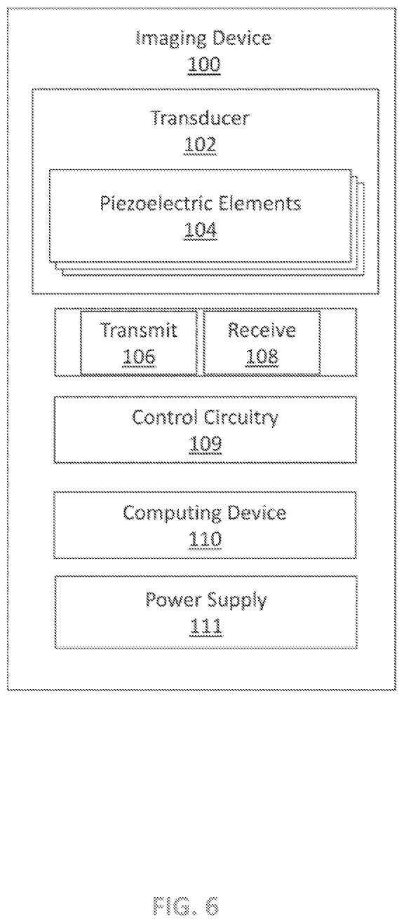

FIG. 6 illustrates a block diagram of an imaging device 100 with transmit 106 and receive channels 108, controlled by control circuitry 109, and a computing device 110 configured to implement various imaging computations. The imaging device 100 may optionally include a power supply 111 to energize the various components of the imaging device 100. In some cases, the computing device 110 and/or the power supply 111 may be external to the imaging device 100.

In some embodiments, the imaging device 100 may be used to generate an image of internal tissue, bones, blood flow, or organs of human or animal bodies. In some cases, the imaging device 100 transmits a signal into the body and receives a reflected signal from the body part being imaged. Such imaging devices 100 may include, for instance, piezoelectric transducers 102, which may also be referred to herein as transceivers or imagers, and which may be based on photo-acoustic or ultrasonic effects. The imaging device 100 can be used to image other objects as well. For example, the imaging device 100 can be used in medical imaging, flow measurements for fluids or gases in pipes, lithotripsy, and localized tissue heating for therapeutic and highly intensive focused ultrasound (HIFU) surgery.

In addition to use with human patients, the imaging device 100 may be used to image internal organs of an animal as well. Moreover, in addition to imaging internal organs, the imaging device 100 may also be used to determine direction and velocity of blood flow in arteries and veins, as well as tissue stiffness, with Doppler mode imaging.

The imaging device 100 may be used to perform different types of imaging. For example, the imaging device 100 may be used to perform one dimensional imaging, also known as A-Scan, 2D imaging, also known as B scan (B-mode), three dimensional (3D) imaging, also known as C scan, and Doppler imaging. The imaging device 100 may be switched to different imaging modes and electronically configured under program control.

To facilitate imaging, the imaging device 100 includes an array of piezoelectric transducers 102, each piezoelectric transducer 102 including an array of piezoelectric elements 104. A piezoelectric element 104 may also include two of more sub-elements, each of which may be configurable in a transmit and/or receive operation. The piezoelectric elements 104 may operate to 1) generate waves (e.g., sound waves or pressure waves) that can pass through the body or other mass and 2) receive reflected waves off the object within the body, or the other mass, to be imaged.

In some examples, the imaging device 100 may be configured to simultaneously transmit and receive ultrasonic waveforms. For example, certain piezoelectric elements 104 may send pressure waves toward the target object being imaged while other piezoelectric elements 104 receive the pressure waves reflected from the target object and develop electrical charges in response to the received waves. The electrical charges may be interpreted and/or processed to generate an image of the target object or a portion thereof.

In some examples, each piezoelectric element 104 may emit or receive signals at a certain frequency, known as a center frequency, as well as the second and/or additional frequencies. Such multi-frequency piezoelectric elements 104 may be referred to as multi-modal piezoelectric elements 104 and can expand the bandwidth of the imaging device 100.

The piezoelectric material that forms the piezoelectric elements 104 may contract and expand when different voltage values at a certain frequency are applied. Accordingly, as voltages alternate between different values applied, the piezoelectric elements 104 may transform the electrical energy (i.e., voltages) into mechanical movements resulting in acoustic energy which is emitted as waves at the desired frequencies. These waves are reflected from a target being imaged and are received at the same piezoelectric elements 104 and converted into electrical signals that are then used to form an image of the target.

To generate the pressure waves, the imaging device 100 may utilize a number of transmit channels 106 and a number of receive channels 108. The transmit channels 106 include a number of components that drive the transducer 102, (i.e., the array of piezoelectric elements 104), with a voltage pulse at a frequency that they are responsive to, causing an ultrasonic waveform to be emitted from the piezoelectric elements 104 towards an object to be imaged. The ultrasonic waveform travels towards the object to be imaged and a portion of the waveform is reflected back to the transducer 102, where the receive channels 108 collect the reflected waveform, convert it to an electrical energy, and process it, for example, at the computing device 110, to develop an image that can be displayed and interpreted by a human or a computer.

In some examples, while the number of transmit channels 106 and receive channels 108 in the imaging device 100 remain constant, the number of piezoelectric elements 104 that they are coupled to may vary. This coupling can be controlled by control circuitry 109. In some examples, a portion of the control circuitry 109 may be distributed in the transmit channels 106 and in the receive channels 108. For example, the piezoelectric elements 104 of a transducer 102 may be formed into a 2D array with N columns and M rows.

In one example, the 2D array of piezoelectric elements 104 may have a number of columns and rows, such as 128 columns and 32 rows. The imaging device 100 may have up to 128 transmit channels 106 and up to 128 receive channels 108. Each transmit channel 106 and receive channel 108 can be coupled to multiple or single piezoelectric elements or sub-elements 104. Depending on the imaging mode, each column of piezoelectric elements 104 may be coupled to a single transmit channel 106 and a single receive channel 108. The transmit channel 106 and receive channel 108 may receive composite signals, which composite signals combine signals received at each piezoelectric element 104 within a respective row or column. In another example, (i.e., during a different imaging mode), individual piezoelectric elements 104 can be coupled to their own transmit channel 106 and their own receive channel 108.

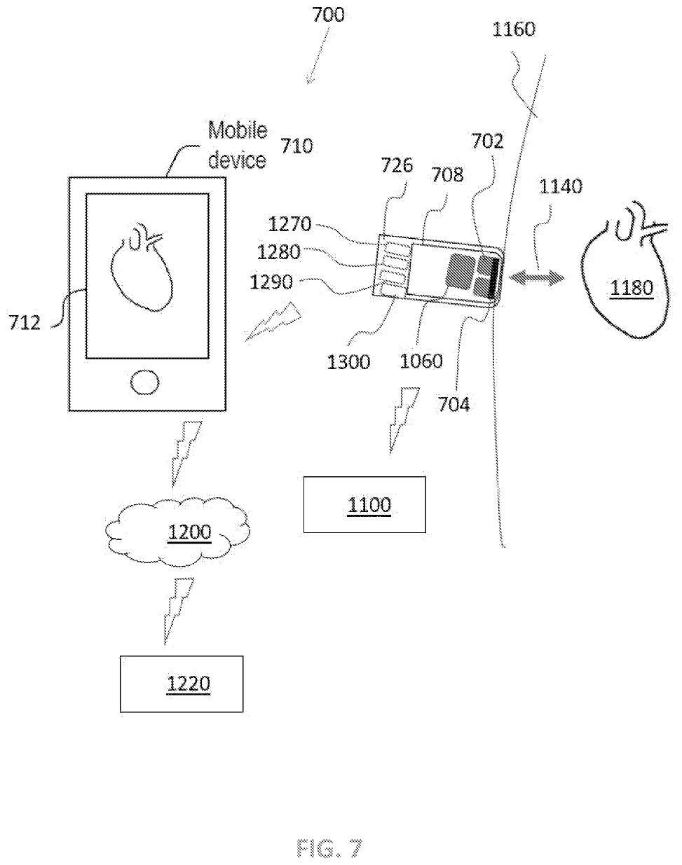

FIG. 7 shows another exemplary embodiment of the ultrasonic imaging system 700 disclosed herein. The imaging system 700 may include a portable device 710 having a display unit 712 and a data recording unit with connection enabled by communication interface to a network 1200, and external databases 1220, such as electronic health records. Such connection to external data sources may facilitate medical billing, data exchange, inquiries, or other medical related information communication. The system 700 may include an ultrasonic imager (also referred to as a “probe” herein) 726 which includes ultrasonic imager assembly (interchangeably referred to as a “tile assembly” herein) 708, where the ultrasonic tile has one or more arrays of pMUTs 702 fabricated on a substrate. The array(s) of pMUTs 702 may be configured to emit and receive ultrasonic waveforms.

The pMUT array 702 may be operatively coupled to (i) an application specific integrated circuit (ASIC) 1060 located in close proximity to the pMUT array 702 and/or (ii) another control unit 1100 located remote from the pMUT array 702. The array may be coupled to impedance lowering and/or impedance matching material 704 which can be placed adjacent to the pMUT array. In some embodiments, the imager 726 includes a rechargeable power source 1270 and/or a connection interface 1280 to an external power source, e.g., a USB interface. In some embodiments, the imager 726 includes an input interface 1290 for an ECG signal for synchronizing scans to ECG pulses. In some embodiments, the imager 726 has an inertial sensor 1300 to assist with user guidance.

In some embodiments, many pMUT arrays can be batch manufactured at low cost. Further, integrated circuits can also be designed to have dimensions such that connections needed to communicate with pMUTs are aligned with each other and pMUT array can be connected to a matching integrated circuit in close proximity, typically vertically below or proximal to the array by a distance, e.g., around 25 μm to 100 μm. Larger arrays of pMUT elements can also be achieved by using multiple pMUT arrays, along with multiple matching ASICs and assembling them adjacent to each other and covering them with appropriate amounts of impedance matching material. Alternately, a single array can have large number of pMUT elements arranged in rectangular arrays or other shapes with a number of pMUT elements ranging from less than 1000 to 10,000. The pMUT array and the plurality of pMUT elements can be connected to matching ASICs.

The arrow 1140 shows ultrasonic transmit beams from the imager assembly 708 targeting a body part 1160 and imaging a target 1180. The transmit beams are reflected by the target being imaged and enter the imager assembly 708 as indicated by arrow 1140. In addition to an ASIC 1060, the imaging system 700 may include other electronic control, communication, and computational circuitry 1100. It is understood that the ultrasonic imager 708 can be one self-contained unit, or it may include physically separate, but electrically or wirelessly connected elements, such as the electronic control unit 1100.

In some cases, the ASIC 1060 can comprise one or more low noise amplifiers (LNA). The pMUTs can be connected to the LNA in receive mode through switches. The LNA converts the electrical charge in the pMUT generated by a reflected ultrasonic beam exerting pressure on the pMUT, to an amplified voltage signal with low noise. The signal to noise ratio of the received signal can be among the key factors that determine the quality of the image being reconstructed. It is thus desirable to reduce inherent noise in the LNA itself. This reduction of noise can be achieved by increasing the transconductance of the input stage of the LNA. This increase in transconductance can be achieved for example by using more current in the input stage. More current may cause power dissipation and heat to increase. However, in cases where low voltage pMUTs are used, with ASIC in close proximity, the power saved by the low voltage pMUTs can be utilized to lower noise in the LNA for a given total temperature rise that is acceptable when compared to transducers operated with high voltage.

In some alternative embodiments, the imager may include a transceiver array for transmitting and receiving pressure waves, and a feature or component for steering the propagation direction of and/or focusing the pressure waves. The imager may also include a control unit, such as an ASIC, for controlling the transceiver array and coupled to the transducer array. The combination of the transceiver array with the ASIC connected to it may constitute a tile. Additional components may include one or more Field Programmable Gate Arrays (FPGAs) for controlling the components of the imager, a circuit(s), such as Analog Front End (AFE), for processing/conditioning signals; and an acoustic absorber for absorbing waves that are generated by the transducer array and propagate toward the circuit. Additional components may optionally include a communication unit for communicating data with an external device through one or more ports; a memory for storing data; a battery for providing a more portable source of electrical power to the components of the imager; and/or a display for displaying a user interface and ultrasound-derived images.

In some instances, during operation of the imager, a user may cause the pMUTs surface, covered by an interface material, to contact a body part area upon which ultrasonic waves are transmitted towards the target being imaged. The imager receives reflected ultrasonic beams from the imaging target and processes or transmits the reflected beams to an external processor for image processing and/or reconstruction, and then to a portable device for displaying an image.

When using the imager, for example to image human or animal body part, the transmitted ultrasonic waveform can be directed towards the target. Contact with the body can be achieved by holding the imager in close proximity of the body, usually after a gel is applied on the body and the imager placed on the gel, to allow superior interface of ultrasonic waves being emitted to enter the body and also for ultrasonic waveforms reflected from the target to reenter the imager, where the reflected signal is used to create an image of the body part and results displayed on a screen, including graphs, plots, statistics shown with or without the images of the body part in a variety of formats.

In some embodiments, the imager/probe may be configured with certain parts being physically separate yet connected through a cable or wireless communications connection. In one example, the pMUT assembly and the ASIC and some control and communications related electronics can reside in a unit often called a probe. The part of the device or probe that contacts the body part may comprise the pMUT assembly.

### Signal Processing Parameters

In some cases, a computing device (e.g., a processor or a computer) may identity or determine the optimal parameters for processing one or more ultrasound signals. The optimal parameters may include, for example, the time delay applied to one or more of the ultrasound signals.

In some cases, the parameters may be detected automatically based on one or more images taken using the imaging device. For instance, the images taken by the imaging device may be analyzed using an image analysis algorithm to determine the optimal parameters. In other cases, the parameters may be detected automatically based on one or more sensor readings or measurements (e.g., from accelerometers or motion sensors operatively coupled to the imaging device or any of the scanning systems described herein). Alternatively, the user of the imaging device may manually input the desired parameters (e.g., time delay values).

### Scanning Parameters

In some embodiments, the scanning parameters controlling an operation of the imaging systems and devices described herein may be adjusted based on the time delay values used. In some cases, the scanning parameters may be controlled or adjusted differently for different imaging operations, different users, and/or different imaging devices.

In some embodiments, the scanning parameter may comprise, for example, the transmit pulse, pulse width, pulse power, pulse repetition interval, pulse order, pulse timing, pulse cycle, transmit frequency, pulse start time, pulse end time, pulse duration, or pulse type (e.g., unipolar pulses, multi-state bipolar pulses), or any other property associated with a pulse that is transmittable by the imaging device. The pulse may comprise an audio or ultrasound signal or wave. In some embodiments, the scanning condition or parameter may comprise pulse frequency, pulse wavelength, phase delay, or phase differences between two or more pulses.

In some embodiments, the scanning parameter may comprise transmit focus depth, or penetration depth. In some embodiments, the scanning parameter may comprise a number of beams, a type of beam (pulsed vs continuous wave), a beam size, a beam geometry (e.g., beam origin, beam directionality, beam elevation/azimuth, or any other property or characteristic of a beam that can be generated and transmitted by the imaging device). The beam may comprise an audio or ultrasound signal or wave.

In some embodiments, the scanning parameter may comprise a scanning mode. The scanning mode may comprise, for example, 2D imaging or 3D imaging. In some embodiments, the scanning parameter may comprise scanning speed, scanning direction, or scanning pattern.

In some embodiments, the scanning parameter may comprise the aperture (e.g., aperture size or aperture shape). In some embodiments, the scanning parameter may comprise the type of filter used, transmit delay, gain, weighting or apodization of signals transmitted or received by individual transducer elements, decimation, line-spacing, or a number of collinear transmits per unit time or per unit area.

Although certain embodiments and examples are provided in the foregoing description, the instant subject matter extends beyond the specifically disclosed embodiments to other alternative embodiments and/or uses, and to modifications and equivalents thereof. Thus, the scope of the claims appended hereto is not limited by any of the particular embodiments described. For example, in any method or process disclosed herein, the acts or operations of the method or process may be performed in any suitable sequence and are not necessarily limited to any particular disclosed sequence. Various operations may be described as multiple discrete operations in turn, in a manner that may be helpful in understanding certain embodiments; however, the order of description should not be construed to imply that these operations are order dependent. Additionally, the structures, systems, and/or devices described herein may be embodied as integrated components or as separate components.

For purposes of comparing various embodiments, certain aspects and advantages of these embodiments are described. Not necessarily all such aspects or advantages are achieved by any particular embodiment. Thus, for example, various embodiments may be carried out in a manner that achieves or optimizes one advantage or group of advantages as taught herein without necessarily achieving other aspects or advantages as may also be taught or suggested herein.

As used herein A and/or B encompasses one or more of A or B, and combinations thereof such as A and B. It will be understood that although the terms “first,” “second,” “third,” etc. may be used herein to describe various elements, components, regions and/or sections, these elements, components, regions and/or sections should not be limited by these terms. These terms are merely used to distinguish one element, component, region or section from another element, component, region, or section. Thus, a first element, component, region, or section discussed below could be termed a second element, component, region, or section without departing from the teachings of the present disclosure.

The terminology used herein is for the purpose of describing particular embodiments only and is not intended to limit the present disclosure. As used herein, the singular forms “a,” “an,” and “the” are intended to include the plural forms as well, unless the context clearly indicates otherwise. It will be further understood that the terms “comprises” and/or “comprising,” or “includes” and/or “including,” when used in this specification, specify the presence of stated features, regions, integers, steps, operations, elements and/or components, but do not preclude the presence or addition of one or more other features, regions, integers, steps, operations, elements, components and/or groups thereof.

As used in this specification and the claims, unless otherwise stated, the term “about,” and “approximately,” or “substantially” refers to variations of less than or equal to +/−0.1%, +/−1%, +/−2%, +/−3%, +/−4%, +/−5%, +/−6%, +/−7%, +/−8%, +/−9%, +/−10%, +/−11%, +/−12%, +/−14%, +/−15%, or +/−20% of the numerical value depending on the embodiment. As a non-limiting example, about 100 meters represents a range of 95 meters to 105 meters (which is +/−5% of 100 meters), 90 meters to 110 meters (which is +/−10% of 100 meters), or 85 meters to 115 meters (which is +/−15% of 100 meters) depending on the embodiments.

While preferred embodiments have been shown and described herein, it will be obvious to those skilled in the art that such embodiments are provided by way of example only. Numerous variations, changes, and substitutions will now occur to those skilled in the art without departing from the scope of the disclosure. It should be understood that various alternatives to the embodiments described herein may be employed in practice. Numerous different combinations of embodiments described herein are possible, and such combinations are considered part of the present disclosure. In addition, all features discussed in connection with any one embodiment herein can be readily adapted for use in other embodiments herein. It is intended that the following claims define the scope of the disclosure and that methods and structures within the scope of these claims and their equivalents be covered thereby.

What is claimed is:

1. An ultrasound imaging system, comprising:
an array of transducer elements;
a plurality of receive circuits configured to provide one or more output signals, wherein the plurality of receive circuits comprise at least one input coupled to at least one transducer element of the array of transducer element;
a plurality of delay circuits configured to output one or more delayed signals, wherein the plurality of delay circuits comprise one or more inputs coupled to at least one receive circuit of the plurality of receive circuits, and wherein the plurality of delay circuits are characterized by a plurality of time delay values; and
at least one multi-channel beamformer configured to (i) receive representations of a plurality of microbeamformed signals and (ii) output at least one representation of a beamformed signal, wherein the plurality of microbeamformed signals represent a combination of delayed signals from the plurality of delay circuits,
wherein the plurality of time delay values are controllable or adjustable such that one or more points-of-focus characterizing the microbeamformed signals move along a line of sight.
